(19) United States
(12) Patent Application Publication
(10) Pub. No.: US 2012/0238002 A1
(43) Pub. Date: Sep. 20, 2012

(54) Photobioreactors Comprising Membrane Carbonation Modules and Uses Thereof

(75) Inventors: Bruce E. Rittman, Tempe, AZ (US); Hyun Woo Kim, Tempe, AZ (US); Andrew Marcus, Scottsdale, AZ (US)

(73) Assignee: ARIZONA BOARD OF REGENTS FOR AND ON BEHALF OF ARIZONA STATE UNIVERSITY, Scottsdale, AZ (US)

(21) Appl. No.: 13/421,449

(22) Filed: Mar. 15, 2012

Related U.S. Application Data
(60) Provisional application No. 61/453,467, filed on Mar. 16, 2011.
(60) Provisional application No. 61/453,882, filed on Mar. 17, 2011.

(51) Int. Cl. C12M 1/42; C12N 1/12

(52) U.S. Cl. 435/257.1; 435/292.1

(57) ABSTRACT

Apparatuses, systems, and methods for using membrane carbonation modules and photobioreactors. Membrane carbonation modules and systems use gas-transfer membranes to supply inorganic carbon for photoautotrophic microorganism growth in a photobioreactor and to withdraw gases from a photobioreactor.

### CROSS-REFERENCE TO RELATED APPLICATIONS

This application claims priority to U.S. Provisional Patent Application Ser. No. 61/453,467 filed Mar. 16, 2011 and U.S. Provisional Patent Application Ser. No. 61/453,882 filed Mar. 17, 2011. These provisional applications are expressly incorporated by reference.

### STATEMENT REGARDING FEDERALLY SPONSORED RESEARCH

This invention was made with government support under ORSPA Account No. KXS 0012 awarded by the Department of Energy and the Advanced Research Projects Agency-Energy. The government has certain rights in the invention.

### BACKGROUND

Carbon dioxide (CO₂) is the major greenhouse gas contributing to global climate change; thus, efforts to reduce CO₂ discharge are needed to minimize and ultimately reverse climate change. Biofuel production from photobioreactors comprising photoautotrophic biomass is a promising energy solution, since CO₂ fixation makes the biofuels carbon neutral.

Maximizing efficiency of a photobioreactor requires matching the nutrient supply rates with the rate of biomass synthesis. Past research, focusing on the natural environment, has emphasized the effects of light, nitrogen, and phosphorus for preventing algal blooms. A key phenomenon that must be understood is the rate at which the photoautotrophic microorganism acquires nutrients, so that its growth can be precisely controlled during photosynthesis. Among the nutrients, inorganic carbon (Cᵢ) presents the largest demand for photoautotrophic growth, since carbon (C) constitutes approximately 50% of biomass dry weight (DW). Particularly in large-scale photobioreactor applications, the Cᵢ supply rate is massive and must be accomplished efficiently.

Controlling the supply of Cᵢ to a photobioreactor is difficult using known techniques. CO₂-gas aeration is the most common approach. When it dissolves in the water, CO₂ gas partitions among its aqueous forms(i.e., Cᵢ in CO_{2(aq)}, HCO₃⁻, and CO₃²⁻) according to the pH. CO₂-gas-aeration approaches can be inefficient because the CO₂ has a tendency to bubble out of the water, rather than dissolving in the water and supplying Cᵢ to the biomass.

Because photoautotrophic microorganisms can selectively take up Cᵢ from CO_{2(aq)} and HCO₃⁻, Cᵢ transfer and its speciation in a photobioreactor affect how Cᵢ is made available to the photoautotrophic organisms, as well as how rate limitation by Cᵢ occurs. Concentrations of Cᵢ species and pH levels can become significant limiting factors for the photoautotrophic growth in a photobioreactor.

Photoautotrophic organisms have an optimal pH at which they thrive. For example, the optimal pH for certain species of photoautotrophic organisms is between pH 7.5 and 9.5. Total Cᵢ and its speciation are critically connected with pH of the growth medium solution, and the pH in photobioreactors often is changed by CO₂ delivery. A challenge, therefore, is finding an efficient way to control the growth of photoautotrophic organisms in a scalable photobioreactor for producing a range of renewable bioproducts.

The shortcomings of CO₂-gas aeration techniques are not intended to be exhaustive, but rather are among many that tend to impair the effectiveness of previously known techniques in the art of bioreactors; however, those mentioned here are sufficient to demonstrate that the methodologies appearing in the art have not been satisfactory and that a significant need exists for the techniques described and claimed in this disclosure.

### SUMMARY OF THE INVENTION

In general, the invention relates to systems comprising a photobioreactor comprising a membrane carbonation module. The photobioreactor typically will comprise at least one vessel comprising a center and light-permitting wall. The membrane carbonation module within the photobioreactor typically will comprise a plurality of hollow fiber membranes, each hollow fiber membrane comprising a membrane wall forming an inner lumen. Such systems are adapted to, during use, comprise a liquid and photoautotrophic organisms suspended in the liquid, with the membrane carbonation module in operable contact with the liquid.

Almost any form of photoautotrophic microorganism can be grown in such a photobioreactor. In some embodiments, the photobioreactor is adapted for the growth of cyanobacteria.

The systems of the invention may further comprise a pressure modulator coupled to the membrane carbonation module. In such systems, each hollow fiber membrane is sealed at one end and the pressure modulator is configured to supply CO₂ to the inner lumens of the hollow fiber membranes. In some cases, the pressure modulator is configured to apply negative pressure to the inner lumen of each hollow fiber membrane such that a gaseous product may be removed from the membrane carbonation module.

The membrane carbonation module may be positioned within the vessel. In some cases where the photobioreactor comprises a light region near the light-permitting wall and a dark region near the center, the membrane carbonation module is positioned within the light region. In other systems, the membrane carbonation module is positioned within the dark region. In some embodiments there is a plurality of membrane carbonation modules and there can optionally be a plurality of pressure modulators coupled to the membrane carbonation modules. Some systems comprise at least one membrane carbonation module in the light region and at least one membrane carbonation module in the dark region.

The system of some embodiments further comprises a recirculation chamber coupled to the photobioreactor and a pump coupled to the photobioreactor and the recirculation chamber, where the pump is configured to circulate a volume of liquid from the vessel, to the recirculation chamber, and back to the vessel. In such systems, the membrane carbonation module can be positioned within the recirculation chamber.

The invention also relates to methods of growing photoautotrophic microorganisms comprising: placing a liquid and photoautotrophic organisms in the photobioreactor of a system as described above; and diffusing gas molecules across the membrane walls of the plurality of hollow fiber membranes. In most cases, these methods further comprise supplying CO₂ gas to the plurality of hollow fiber membranes using the pressure modulator and diffusing CO₂ molecules across each membrane wall from the inner lumen to the liquid. In some embodiments, the methods further comprise adjusting the rate at which CO₂ gas is supplied to the plurality of hollow fiber membranes until a desired pH level is reached. In these embodiments monitoring the pH level and adjusting the rate at which CO₂ gas is supplied can be accomplished by software.

Typically, the methods of the invention comprise extracting bio-derived products and/or photoautotrophic organisms from the photobioreactor.

In addition to putting CO₂ into the bioreactor, methods of the invention can further comprise removing gases from the bioreactor by diffusing a gaseous product through each membrane wall from the liquid to the inner lumens of the hollow fiber membranes. In such cases, a negative pressure may be applied to the plurality of hollow fiber membranes with the pressure modulator, allowing for the collection of the gaseous product. In some embodiments the gas can be H₂, O₂, and/or N₂.

The invention also relates to any system as described above for use in the growth of growing photoautotrophic organisms.

The term “coupled” is defined as connected, although not necessarily directly, and not necessarily mechanically.

The terms “a” and “an” are defined as one or more unless this disclosure explicitly requires otherwise.

The term “substantially” and its variations are defined as being largely but not necessarily wholly what is specified as understood by one of ordinary skill in the art, and in one non-limiting embodiment “substantially” refers to ranges within 10%, preferably within 5%, more preferably within 1%, and most preferably within 0.5% of what is specified.

The terms “comprise” (and any form of comprise, such as “comprises” and “comprising”), “have” (and any form of have, such as “has” and “having”), “include” (and any form of include, such as “includes” and “including”) and “contain” (and any form of contain, such as “contains” and “containing”) are open-ended linking verbs. As a result, a method or device that “comprises,” “has,” “includes” or “contains” one or more steps or elements possesses those one or more steps or elements, but is not limited to possessing only those one or more elements. Likewise, a step of a method or an element of a device that “comprises,” “has,” “includes” or “contains” one or more features possesses those one or more features, but is not limited to possessing only those one or more features. Furthermore, a device or structure that is configured in a certain way is configured in at least that way, but may also be configured in ways that are not listed.

Other features and associated advantages will become apparent with reference to the following detailed description of specific embodiments in connection with the accompanying drawings.

The schematic flow chart diagrams that follow are generally set forth as logical flow chart diagrams. As such, the depicted order and labeled steps are indicative of one embodiment of the presented method. Other steps and methods may be conceived that are equivalent in function, logic, or effect to one or more steps, or portions thereof, of the illustrated method. Additionally, the format and symbols employed are provided to explain the logical steps of the method and are understood not to limit the scope of the method. Although various arrow types and line types may be employed in the flow chart diagrams, they are understood not to limit the scope of the corresponding method. Indeed, some arrows or other connectors may be used to indicate only the logical flow of the method. For instance, an arrow may indicate a waiting or monitoring period of unspecified duration between enumerated steps of the depicted method. Additionally, the order in which a particular method occurs may or may not strictly adhere to the order of the corresponding steps shown.

All of the apparatuses, systems, and methods disclosed and claimed herein can be made and executed without undue experimentation in light of the present disclosure. While the apparatus and methods of this invention have been described in terms of preferred embodiments, it will be apparent to those of skill in the art that variations may be applied to the methods and in the steps or in the sequence of steps of the method described herein without departing from the concept, spirit and scope of the invention. In addition, modifications may be made to the disclosed apparatus and components may be eliminated or substituted for the components described herein where the same or similar results would be achieved. All such similar substitutes and modifications apparent to those skilled in the art are deemed to be within the spirit, scope, and concept of the invention as defined by the appended claims.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1 is an embodiment of a system comprising a membrane carbonation module and a photobioreactor where the membrane carbonation module is positioned in the dark region of the photobioreactor.

FIG. 2 is an embodiment of a membrane carbonation module comprising a plurality of hollow fiber membranes.

FIG. 3 is a partial cross-section view of a hollow fiber membrane showing gas diffusion into the surrounding liquid from the hollow fiber membrane.

FIG. 4 is a plot of H₂CO₃, HCO₃ and CO₃²⁻ as a function of pH level.

FIG. 5 is a plot of generalized molecules P⁻, HP, and H₂P⁺ as a function of pH level.

FIG. 6 is a partial cross-section view of a hollow fiber membrane showing gas diffusion from the surrounding liquid into the hollow fiber membrane.

FIG. 7 is a partial cross-section view of a hollow fiber membrane showing the filtration capabilities of a hollow fiber membrane.

FIG. 8 is a schematic plot of light intensity and biomass growth as a function of distance from a light source.

FIG. 9 is a schematic plot of CO₂ concentration as a function of dispersion and distance from the photobioreactor wall.

FIG. 10 is a schematic plot of pH levels, CO₂ concentration, and biomass concentration as a function of distance from the photobioreactor wall in a high dispersion case.

FIG. 11 is a schematic plot of pH levels, CO₂ concentration, and biomass concentration as a function of distance from the photobioreactor wall in a low dispersion case.

FIG. 12 is one embodiment of a system where the membrane carbonation module is positioned in the light region of the photobioreactor.

FIG. 13 is a schematic embodiment of a photobioreactor comprising multiple membrane carbonation modules.

FIG. 14 is a schematic plot showing the CO₂ concentration in a photobioreactor like that in FIG. 13.

FIG. 15 is one embodiment of a system where the membrane carbonation module is positioned in a recycling chamber coupled to the photobioreactor.

FIG. 16 is a schematic flow chart for one method of using the system to adjust the pH level in the liquid.

FIG. 17 is one embodiment of a system used in the proof of concept.

FIG. 18 is partial cross-section view of one embodiment of a hollow fiber membrane used in the proof of concept.

FIG. 19 is a plot of dynamics of biomass (as DW), Cᵢ species, and pH as Q_{R} was increased from 24 to 73 L/d. Fixed were the CO₂ pressure of 1 atm, HRT of 5 d, A_{M} of 44 cm², and LI of 44 W/m² as PAR.

FIG. 20 is a plot of biomass production rate.

### DETAILED DESCRIPTION

Various features and advantageous details are explained more fully with reference to the non-limiting embodiments that are illustrated in the accompanying drawings and detailed in the following description. Descriptions of well known starting materials, processing techniques, components, and equipment are omitted so as not to unnecessarily obscure the invention in detail. It should be understood, however, that the detailed description and the specific examples, while indicating embodiments of the invention, are given by way of illustration only, and not by way of limitation. Various substitutions, modifications, additions, and/or rearrangements within the spirit and/or scope of the underlying inventive concept will become apparent to those skilled in the art from this disclosure.

A photobioreactor uses microorganisms to generate valuable biomass or bio-derived products. Photoautotrophic microorganisms, the main target organisms for a photobioreactor, require inorganic carbon (Cᵢ) for growth or to produce the bio-derived product. Rapid growth of microorganisms or rapid production of the bio-derived product requires that the Cᵢ-supply match the growth rate or the production rate.

A membrane carbonation module is disclosed that may be used to deliver Cᵢ directly to the phototropic microorganisms in the form of gaseous CO₂. The disclosed membrane carbonation module may be used to control distributions of pH, gaseous and aqueous solutes (e.g., CO₂ and HCO₃⁻), biomass, and biomass-derived products with targeted delivery of gaseous substrates to and/or removal of gaseous products from a photobioreactor.

A photobioreactor comprises a vessel that contains a photoautotrophic biomass suspended in a liquid. Most commonly, a photobioreactor grows photoautotrophic microorganisms that require Cᵢ for growth. One or more membrane carbonation modules can be combined with a photobioreactor to make a membrane carbonation photobioreactor.

The one or more membrane carbonation modules are wetted by the liquid. In some embodiments, one or more membrane carbonation modules may be positioned inside the photobioreactor. In other embodiments, one or more membrane carbonation modules may be positioned within a photobioreactor-associated compartment, such as a recirculation chamber. Positioning the membrane carbonation module inside or outside the photobioreactor spatially couples or decouples important tasks such as growing microorganisms, adjusting pH, recovering products, and removing gases.

For example, placing a membrane carbonation module in a recirculation chamber spatially decouples CO₂ supply from where the microbes grow. By taking advantage of this decoupling, bio-derived products may be recovered inside the photobioreactor or in the recirculation chamber, depending on which embodiments are used.

A system may be operated to provide an environment where microorganisms perform desirable material transformation (e.g., generation of biomass, generation of biomass-derived chemicals, and removal of chemicals). Material transformation depends on many parameters, including biomass, light intensity, nutrient availability (e.g., Cᵢ, nitrogen, and phosphorus), and pH. Distribution of these parameters within a vessel may be uniform or non-uniform. Membrane carbonation modules may be positioned in various configurations to achieve controlled delivery of gases (e.g., CO₂) or removal of gases (e.g., O₂, H₂, and/or N₂) to precisely control the rate of microbiological material transformation. Because the membrane carbonation modules can operate without gravity, the technology also can be used in space travel.

### Embodiments of Membrane Carbonation Module Photobioreactor Systems

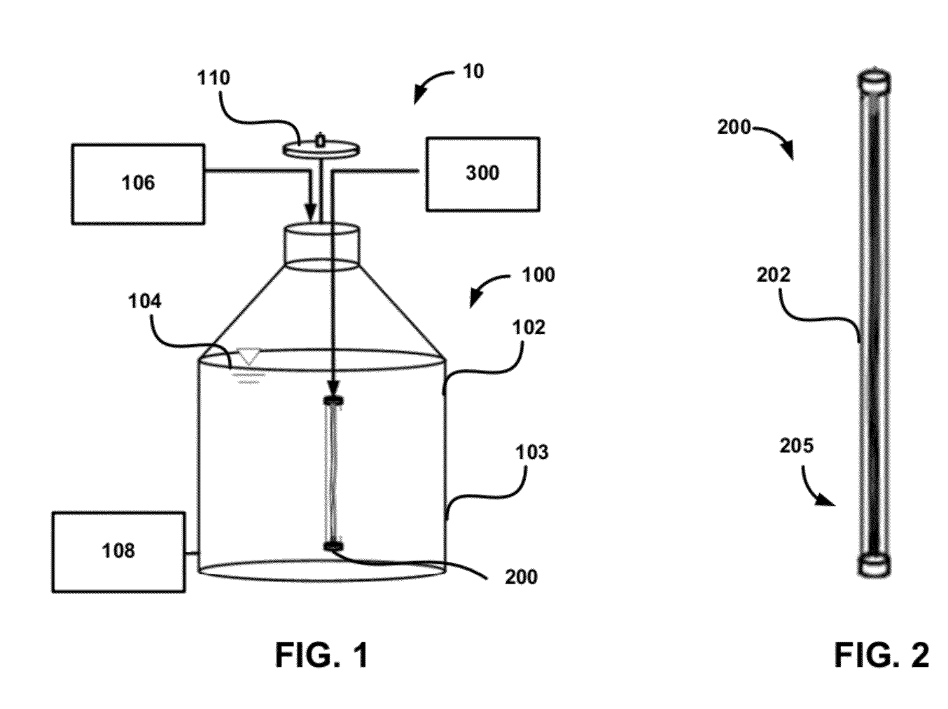

FIG. 1 shows a schematic diagram of one embodiment of a system 10 (i.e., a membrane carbonation module photobioreactor) comprising a photobioreactor 100 and a membrane carbonation module 200. In the embodiment shown, photobioreactor 100 comprises a vessel 102 that contains a liquid 104. The liquid 104 comprises a suspension of biomass. In certain embodiments, the biomass may comprise photoautotrophic microorganisms such as cyanobacteria or algae. In certain embodiments, cyanobacterium Synechocystis sp. strain PCC6803 may be used.

In the illustrated embodiment, vessel 102 comprises a wall 103 comprising a light-permitting material. In other embodiments, vessel 102 comprises at least one light-permitting wall 103 or light-permitting panel. In the embodiments illustrated here, vessel 102 is a vertical cylinder, though any suitable shape may be used. For example, vessel 102 may be a horizontal cylinder or substantially spherical in certain embodiments. In other embodiments, vessel 102 may be a cube or a rectangular prism.

System 10 also comprises an inlet pump 106 and an exit pump 108. Inlet pump 106 supplies fluid to vessel 102. Exit pump 108 removes liquid 104 from vessel 102. In some embodiments, photobioreactor 100 further comprises a vent 110 to permit the venting of gases, such as O₂ and/or H₂.

In the illustrated embodiment, one membrane carbonation module 200 is shown submerged in liquid 104 in vessel 102. Other configurations will be discussed in more detail below. As shown in detail in FIG. 2, membrane carbonation module 200 comprises a plurality of hollow fiber membranes 202. In the illustrated embodiments, hollow fiber membranes 202 comprise tubular membranes having a circular (i.e., annular) cross-section, though membranes that are flat or ribbon-shaped may also be used.

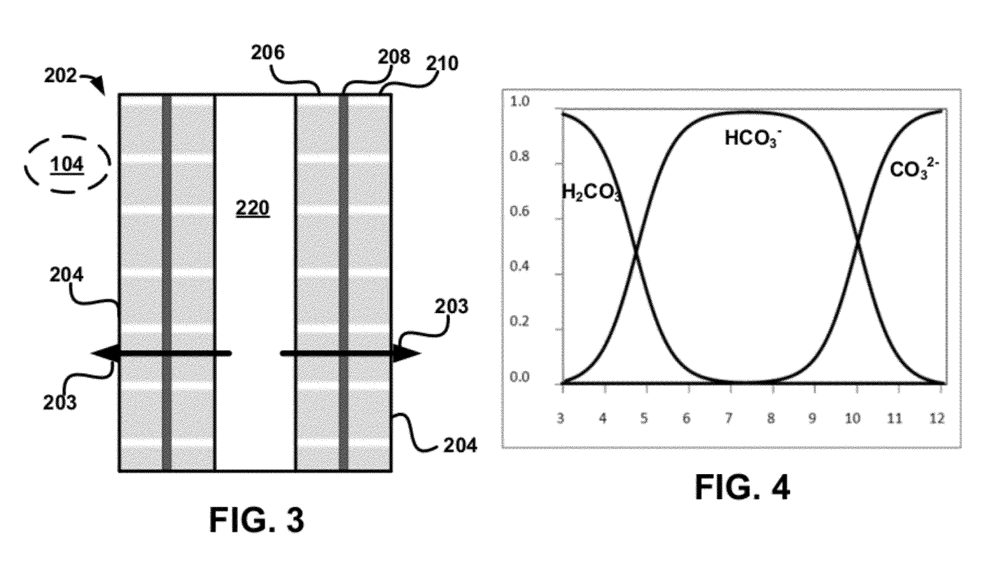

FIG. 3 illustrates a side cross-section view of a hollow-fiber gas-transfer membrane 202. In the embodiment shown, hollow fiber membrane 202 comprises a membrane wall 204 that forms an inner lumen 220. Membrane wall 204 comprises a microporous or nonporous material that prevents liquid from flowing through the wall and into the inner lumen. In the illustrated embodiment, membrane wall 204 comprises three layers: an inner layer 206, a middle layer 208, and an outer layer 210. In the embodiment shown, inner layer 206 and outer layer 210 comprise hydrophobic microporous polyethylene, while the middle layer 208 comprises dense polyurethane. Each hollow fiber membrane 202 may be sealed at an end 205 to allow for the application of a positive pressure or a negative pressure to inner lumen 220.

Pressure modulator 300 is coupled to membrane carbonation module 200. Pressure modulator 300 may be a pump coupled to gas source (e.g., a CO₂ supply) or a negative pressure source (e.g., a vacuum pump). In one embodiment, pressure modulator 300 supplies CO₂ gas to inner lumens 220 of the plurality of hollow fiber membranes 202 that have been immersed in liquid 104. As shown by arrows 203, the CO₂ molecules may then diffuse across membrane wall 204 from an area of high concentration (i.e., within inner lumen 220) to an area of low concentration (i.e., in liquid 104).

In another embodiment, gaseous products (e.g., O₂, H₂, and/or N₂) may be present in high concentrations within liquid 104. These gaseous products may diffuse across membrane wall 204 into inner lumens 220 of the plurality of hollow fiber membranes 202. Pressure modulator 300 may be configured to apply a negative pressure to inner lumens 220 of the plurality of hollow fiber membranes 202, removing the gaseous products from hollow fiber membranes 202 and recovering them for storage.

In each case, gas transfer across membrane wall 204 may continue until the concentration in liquid 104 and the concentration in inner lumen 220 reach equilibrium.

### Use of Membrane Carbonation Module to Control pH Level in Photobioreactor

Membrane carbonation module 200 also may be used to control the pH level in liquid 104. When CO₂ is delivered to liquid 104, it partitions into CO_{2(aq)}, HCO₃⁻, and CO₃²⁻ according to the pH level of liquid 104. HCO₃⁻ and CO₃²⁻ are normally the main alkalinity species controlling the pH inside a photobioreactor.

As shown in FIG. 4, the carbonate system has pKₐ₁ of 6.3 and pKₐ₂ of 10.3; therefore, H₂CO₃ is dominant below pH<6.3, HCO₃⁻ is dominant between pH 6.3 and 10.3, and CO₃²⁻ is dominant above pH>10.3.

Because certain photoautotrophic organisms can selectively take up only CO_{2(aq)} and HCO₃⁻, CO₂ transfer and its speciation in a photobioreactor affect how CO₂ is made available to photosynthetic organisms and how rate limitation by CO₂ occurs. Thus, pH and concentrations of C₁ species can significantly limit photoautotrophic growth in a photobioreactor.

The pH inside a photobioreactor is set by balance of microbiological growth and CO₂ supply. Microbiological growth can raise pH by consuming HCO₃⁻ and CO_{2(aq)} and by letting OH⁻ represent a larger fraction of alkalinity. CO₂ supply can lower pH by resupplying CO_{2(aq)} into the system and by letting HCO₃⁻ and CO_{2(aq)} represent a larger fraction of alkalinity.

Thus, pH rises when growth exceeds CO₂ supply, and pH lowers when growth lags behind CO₂ supply. Therefore, it is possible to achieve a desired pH at specific location by controlling the rate of CO₂ supply with a membrane carbonation module 200 and a pressure modulator 300.

As discussed above, photoautotrophic microorganisms in photobioreactor 100 produce valuable bio-derived product. The pH in liquid 104 is one of the most important parameters in recovering these products from the photobioreactor 100.

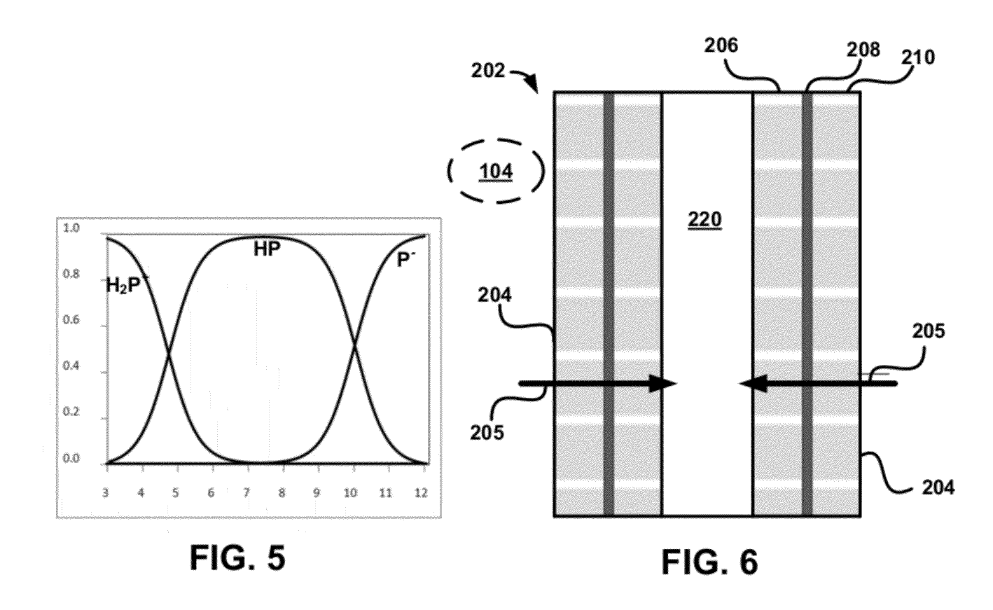

FIG. 5 illustrates the effects of pH control. For example, consider a general bio-derived product H₂P⁺ with pKa₁=4.7 for forming HP and pKa₂=10 for forming P⁻. The anion P⁻ dominates above pH>10, the neutral species HP dominates between pH 4.7 and 10, and the cation H₂P⁺ dominates below pH 4.7. Polar solvents or hydrophilic (anion and cation) resins are commonly used for extracting anion and cations. Hydrophobic solvents and resins are commonly used for extracting hydrophobic (neutral) compounds.

The method used for extraction must be tailored specifically to the molecular structure of each byproduct, since anions that contain hydrophobic moieties (e.g., aromatic rings) can become hydrophobic, and neutral species that contain polar functional group (e.g., alcohol) can be hydrophilic (e.g., ethanol). Membrane carbonation module 200 may be used to adjust pH to a level appropriate for extracting each bio-product at regions in photobioreactor 100 designated for product recovery.

Use of Membrane Carbonation Module to Remove Gaseous Products from Photobioreactor

Photoautotrophic microorganisms in photobioreactor 100 can produce gaseous products, such as O₂, H₂, and N₂, as metabolic byproducts. Removal of these gaseous products from photobioreactor 100 can be desirable for avoiding formation of gas bubbles, for preventing product inhibition, and for recovering valuable product. For example, H₂ has a commercial value as an energy carrier and for chemical synthesis, while O₂ can create an oxic environment in photobioreactor 100 that can inhibit growth of photoautotrophic organisms.

Gaseous products may be removed from photobioreactor 100 using a variation of membrane carbonation module 200. In one embodiment, as shown in FIG. 6, this is achieved by using pressure modulator 300 to apply a negative pressure to inner lumens 220 of hollow fiber membranes 202. Gaseous products, such as O₂, H₂, or N₂, diffuse from an area of high concentration in liquid 104 across membrane wall 204 to an area of low concentration inside inner lumen 220, as shown by arrows 205. The gaseous products may then be removed from the plurality of hollow fiber membranes 220 collected and stored.

### Use of Membrane Carbonation Modules to Achieve a Closed System in a Photobioreactor

One or more membrane carbonation modules 200 may be used to achieve a closed system in photobioreactor 100. One or more membrane carbonation modules 200 may be configured to supply CO₂ to photobioreactor 100, while one or more membrane carbonation modules 200 may be configured to remove gaseous byproducts.

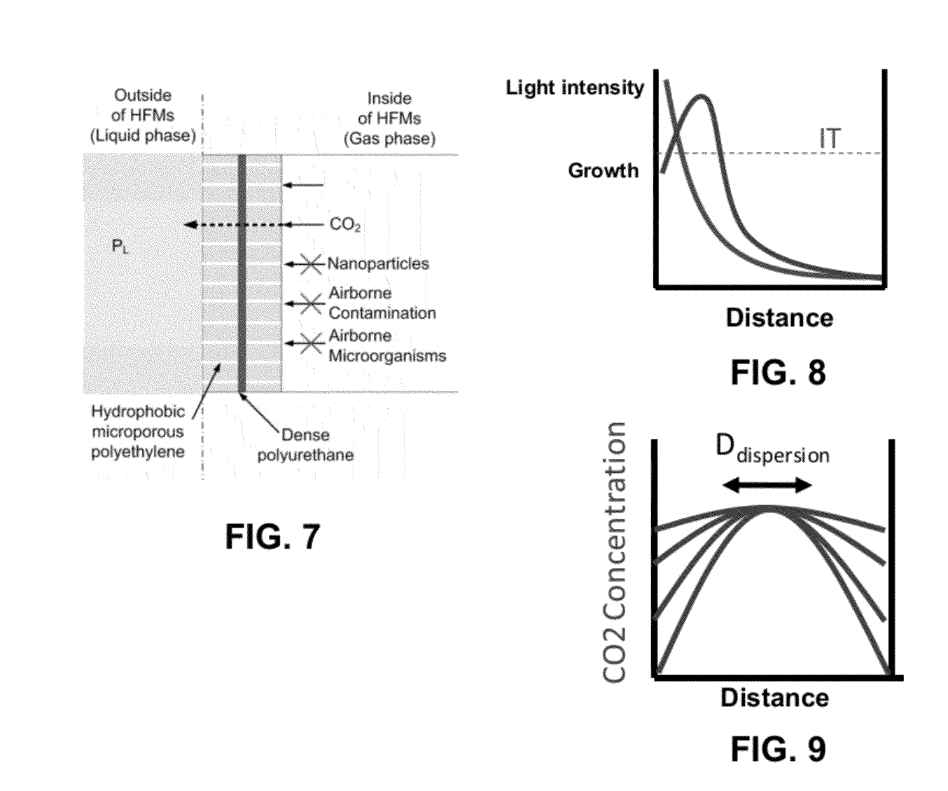

In embodiments where photobioreactor 100 is a closed system, it is often desired to grow a strain of photoautotrophic organisms that is as pure as possible. Use of membrane carbonation module 200 prevents microbiological contamination, as shown in FIG. 7.

Membrane carbonation module 200 prevents contamination by securing the gas inlet and the gas outlet. The gas (e.g., CO₂) entering photobioreactor 100 is a potential source of microbiological contamination. Each hollow fiber membrane 202 has a microporous or non-porous structure that can act as a filter to prevent passage of any of these microorganisms. In addition, a ventilation filter (not shown) may be placed between the pressure modulator 300 and the hollow fiber membrane 202 to duplicate protection.

Ordinarily, when gaseous bubbles (e.g., O₂, H₂, and/or N₂) are removed from a photobioreactor, turbulence caused by ventilating gas into the atmosphere can introduce contaminants from the atmosphere surrounding the vent. Membrane carbonation module 200 avoids this problem because it provides CO₂ on demand, which prevents the formation of gas bubbles. The gas bubbles can, however, form when the metabolic byproducts from photoautotrophic microorganisms accumulate (e.g., O₂, H₂, and/or N₂). In this case, pressure modulator 300 can be used to provide a negative pressure to membrane carbonation module 200, which will allow the gaseous products to diffuse through the membrane. Thus, the membrane carbonation module achieves reactor closure by using hollow fiber membranes for gas transfer and by providing CO₂ on demand.

### Placement of Membrane Carbonation Module in Dark Region or Light Region of Photobioreactor

FIGS. 1 and 8-11 illustrate one embodiment of a membrane carbonation module 200 positioned in a photobioreactor 100. In the illustrated embodiment, vessel 102 comprises a light-permitting material. According to Beer's law, light intensity within vessel 102 decreases exponentially as a function of distance from the light source (i.e., vessel wall 103), as shown in FIG. 8.

According to bacterial kinetics, rapid bacterial growth occurs in the light region near the light source above the inhibition threshold (IT). Little to no bacterial growth occurs in the dark region below the IT threshold.

Referring to FIG. 1, membrane carbonation module 200 is located in the dark region of vessel 102, at or near the center of vessel 102 and away from wall 103 and the light. Locating the membrane carbonation module in the dark region decouples biomass growth from the carbon source, which discourages biofilm formation on the module.

FIG. 9 illustrates CO₂ distributions as a function of distance from the reactor wall. A different CO₂ distribution can result from mass-transfer limitations. Non-homogenous CO₂ distributions can be advantageous, as discussed below.

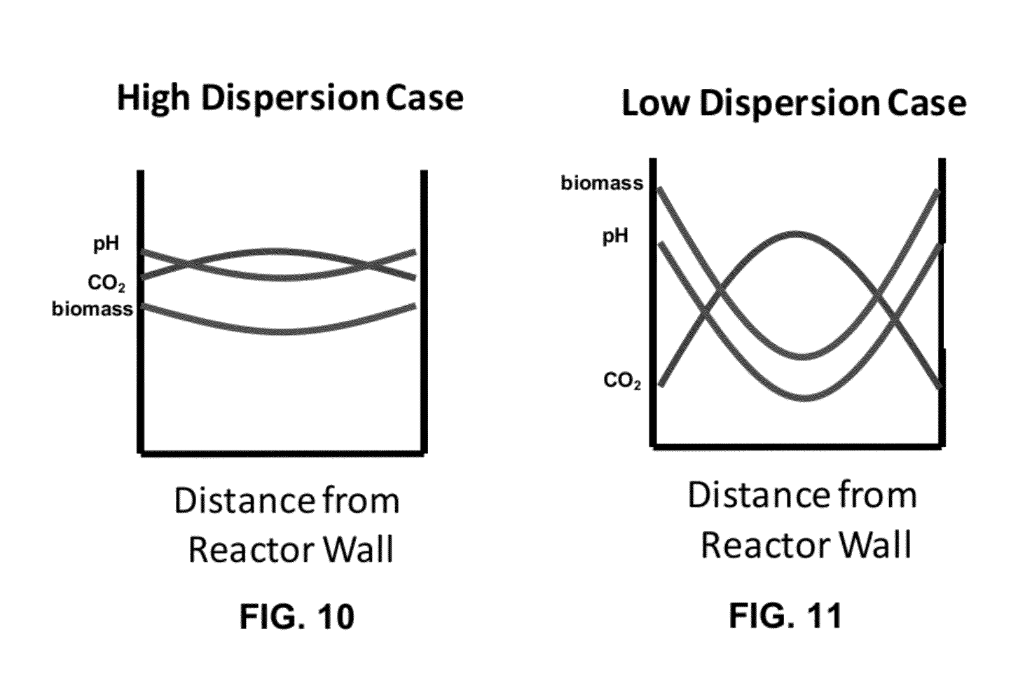

Good mixing is desirable in creating a homogenous distribution of materials in a photobioreactor 100, as shown in FIG. 10. Dispersion of inorganic carbon Cᵢ can be promoted by mechanical mixing (e.g., magnetic stir bar) or by using a pump.

Minimal mixing may be desirable (or can be an inevitable consequence in embodiments where a long tubular vessel is used) in creating a heterogeneous distribution of materials in a photobioreactor 100, as shown in FIG. 11. When membrane carbonation module 200 is located in the dark region of photobioreactor 100 away from wall 103, a combination of low light intensity and low pH can discourage biofilm formation on the module. When dispersion in liquid 104 is high, a stagnant film layer on the surface of hollow fiber membranes 202 can provide enough concentration gradient for discouraging biofilm formation.

Heterogeneity in the chemical distribution can be useful for product recovery. For example, a high pH near wall 103 is suitable for growing photoautotrophic organisms. A low pH in the interior can protonate bio-products and convert them into neutral or acidic hydrophobic form. These bio-products can be recovered using ion-exchange resigns or solvents, as discussed above. Thus, different regions of photobioreactor 100 can be tailored for specific purposes (in this embodiment, biomass growth and bio-product recovery).

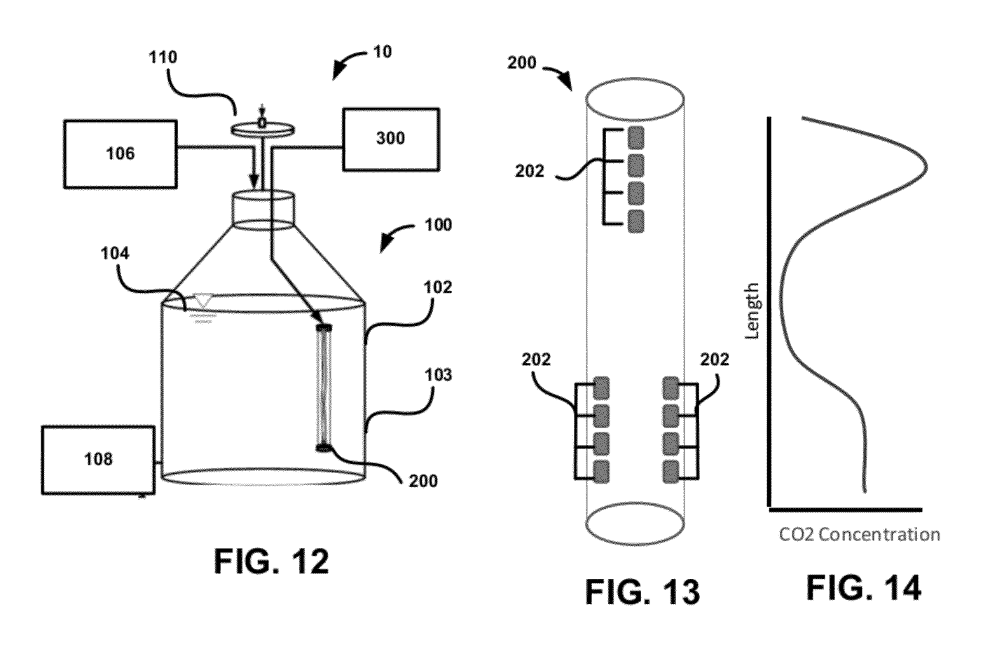

In other embodiments, membrane carbonation module 200 may be placed in the light region near wall 103 of vessel 102, as shown in FIG. 12. As discussed above in reference to FIG. 8, maximum growth of phototrophic organisms is a function of light intensity. Light intensity decreases exponentially as a function of distance from the light source. By placing membrane carbonation module 200 in the light region, a region of high CO₂ and high light intensity is created that increases the rate of product synthesis and encourages biofilm formation.

Membrane Carbonation Module Used to Decouple Delivery of Co₂ from Photobioreactor Orientation

In other embodiments, membrane carbonation modules 200 may be used with a photobioreactor 100 to decouple the orientation of the photobioreactor from delivery of CO₂. In certain embodiments, the photobioreactor is a long cylinder that may be oriented vertically or horizontally.

FIG. 13 shows a schematic example of an embodiment of a vertically-oriented cylindrical photobioreactor 100 that uses multiple membrane carbonation modules 200 to provide for the controlled delivery of CO₂ to specific regions of photobioreactor 100. In this embodiment, membrane carbonation modules 200 are placed in the light region near the bottom of photobioreactor 100, and in the dark region at the top 130 of photobioreactor 100. FIG. 14 shows that the CO₂ concentration at top and bottom of photobioreactor 100 is higher than in the middle of photobioreactor 100. Unlike using aeration techniques, use of membrane carbonation modules 200 can provide for the controlled delivery of CO₂ anywhere within the photobioreactor module to create any desired CO₂ profile.

Membrane Carbonation Module Used to Decouple Co₂ Delivery from Biomass Growth Site

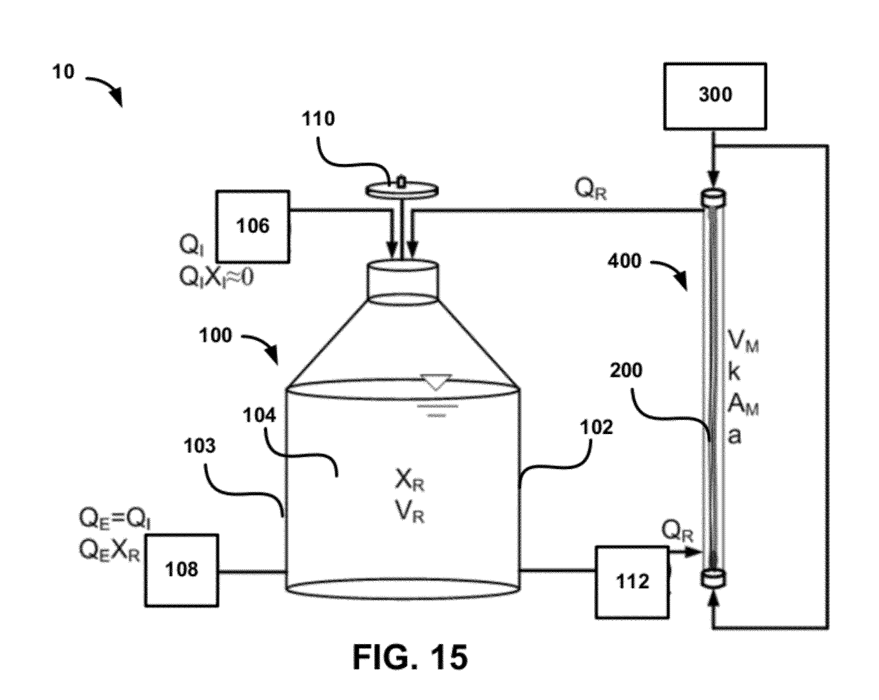

In other embodiments, membrane carbonation modules 200 may be placed in a recirculation chamber 400 separate from but coupled to photobioreactor 100. FIG. 15 shows a schematic example of one such embodiment, where recirculation chamber 400 comprising membrane carbonation module 200 is coupled to photobioreactor 100 via a pump 112. Pump 112 is configured to recirculate liquid 104 from photobioreactor 100, through recirculation chamber 400 and back to photobioreactor 100.

Placing membrane carbonation module 200 in a recirculation loop spatially decouples the delivery of CO₂ supply from where the photoautotrophic microorganisms grow. The concentration of CO₂ inside the photobioreactor can be set by the solid retention time (SRT) and a presence of a rate-limiting factor, which can be light, CO₂, H⁺, or other nutrients. In embodiments where photobioreactor 100 is configured to operate as a chemostat, the CO₂ concentration inside photobioreactor 100 is set at the effluent concentration, which can be the same or different from the concentration in recirculation chamber 400. Thus, microorganisms can see significantly lower concentration of CO₂ in photobioreactor 100, while recirculation chamber 400 may provide an amount of CO₂ equal to the CO₂ utilization in photobioreactor 100.

A low CO₂ concentration in photobioreactor 100 may be useful for three reasons. First, a low CO₂ creates an alkaline pH that many phototrophs prefer. Second, a low CO₂ may activate intracellular carbon concentrating mechanisms. For example, most cyanobacteria have mechanisms to store Cᵢ a low-CO₂ environment. Third, as discussed above, the pH influences recovery of bio-derived products.

The mass balance equation for the difference in CO₂ concentration inside the recirculation chamber 400 and inside the photobioreactor 100 is as follows:

ΔC_{i,T}·Q_{R}=K·A_{M}·(P_{M}−P_{L})=K·A_{M}·(P_{M}−α₀·C_{i,T}·K_{H,pc}).

The driving force for CO₂ delivery inside recirculation chamber 400 is the difference in partial pressure (atm) between gaseous CO₂ inside inner lumen 202 of hollow fiber membranes 220 (P_{M}) and the pressure of CO₂ that would be in equilibrium with the concentration of aqueous CO₂ inside the chamber 400 (P_{L}). P_{L} can be expressed as the concentration of CO₂ in the liquid phase using Henry's law constant K_{H,pc} and the ionization factor (α₀).

Two design parameters, K (L⁻³ atm/M) and membrane surface area (A_{M}), set the capacity of the membrane carbonation module to supply CO₂. Gas-transfer capacity may be increased by adding more hollow fiber membranes 220 to increase A_{M}. In illustrated embodiments, each membrane carbonation module 200 comprises 25 hollow fiber membranes 220. Other embodiments may use more or fewer hollow fiber membranes 220 depending on the desired performance characteristics of the membrane carbonation module 200.

During reactor operation, microorganisms set the CO₂ demand by reducing the CO₂ concentration to the effluent concentration in photobioreactor 100 C_{i,T} (M L⁻³). The magnitude of response by the membrane carbonation module 200 can be controlled by adjusting the recycle flow rate Q_{R} (L³/d) and P_{M}. Having two operational controls may be useful when balancing tradeoffs among different objectives: e.g., pH control, biomass generation, and product recovery.

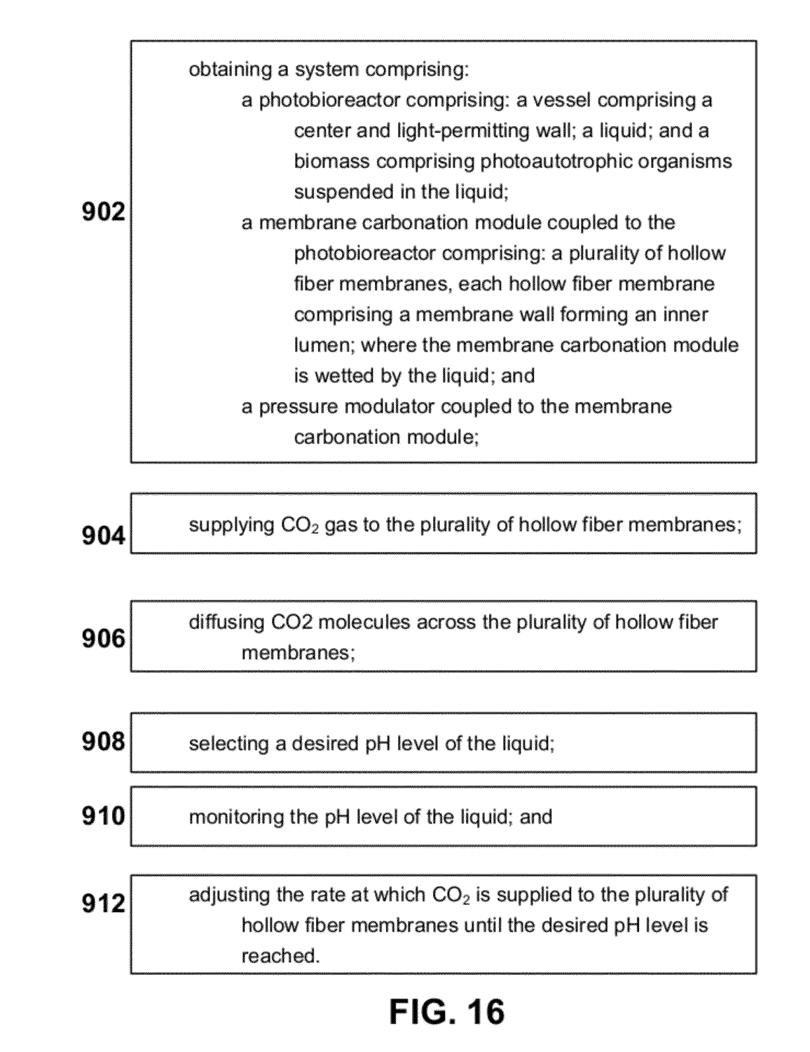

FIG. 16 illustrates a flow chart for a method of supplying CO₂ to a photobioreactor. In disclosed embodiments, the pH level may be measured and/or monitored at various regions within photobioreactor 100 using known methods, such as pH sensors and software configured to implement the method. In some embodiments, a pH sensor or plurality of pH sensors may deployed in various regions of photobioreactor 100. The pH sensors may be used in a feedback loop together with a computer and/or software to maintain desired pH levels within photobioreactor 100. For example, system 10 may be configured such that a membrane carbonation module may increase the CO₂ pressure, which would increase the rate at which CO₂ is delivered, changing the pH level.

In addition, other values may be monitored as well. For instance, system 10 may be configured such that gaseous products (such as H₂ and/or O₂) are automatically removed from photobioreactor 100 upon reaching a certain concentration.

### EXAMPLE

Synechocystis PCC6803 can take up Cᵢ only from CO_{2(aq)} and HCO₃⁻. Therefore, Cᵢ transfer and its speciation in a photobioreactor affect how Cᵢ is made available to PCC6803 and how rate limitation by Cᵢ occurs. pH and concentrations of Cᵢ species can become significant limiting factors for the photoautotrophic growth in a photobioreactor. The optimal pH for PCC6803 is between pH 7.5 and 9.5; thus, PCC6803, in general, prefers slightly alkaline pH, as do other cyanobacteria, although the kinetics of PCC6803 under pH limitation needs better quantification. Recent research demonstrated that PCC6803 has a Monod half-maximum-rate concentration for C₁ of K_{S}=0.5 mgC/L when other nutrients are sufficient. Total Cᵢ and its speciation are critically connected with pH of the growth medium solution, and the pH in photobioreactors often is controlled by the CO₂ delivery rate. A challenge, therefore, is finding an efficient way to control the growth of PCC6803 in a scalable photobioreactor for producing a range of renewable bioproducts. The same principles apply to the wide range of microbial phototrophic organisms besides PCC6803.

The supply rate of Cᵢ to the main photobioreactor was controlled by regulating the recirculation rate (Q_{R}) between the membrane carbonation module and the photobioreactor. The effect of Q_{R} on CO₂ mass transport in membrane carbonation module was evaluated, as well as how it affects the biomass production rate, Cᵢ concentration, and pH in the photobioreactor. The biomass production rate and Cᵢ concentration increased in response to the Cᵢ supply rate (controlled by Q_{R}), but not in the same proportion. The biomass production rate increased less than Cᵢ due to increased light limitation, and the higher Cᵢ concentration caused the pH to decrease. The results demonstrate that a membrane carbonation module offers independent control over the photoautotrophic growth of suspended PCC6803 biomass with minimal loss of CO₂ to the atmosphere in a photobioreactor.

The membrane carbonation module photobioreactor used in the proof of concept uses hollow fiber membranes pressurized with pure CO₂ to deliver Cᵢ to a photobioreactor with PCC6803. A membrane carbonation module photobioreactor system consisting of two compartments was used: a main photobioreactor and a membrane carbonation (membrane carbonation module) chamber, connected with internal recirculation system and a CO₂ source having a variable supply rate. The CO₂ partial-pressure difference between the inside and the outside of each membrane controls the diffusion of gaseous CO₂ (i.e., CO_{2(g)} into the recirculation liquid, which increases the concentration of Cᵢ. The surface area of the hollow fiber membranes contacting the recirculation liquid also controls the rate of CO₂ transfer by the membrane module. For a given CO₂ pressure and hollow fiber membrane surface area, the overall Cᵢ supply rate is determined by the recirculation rate of carbonated liquid, which contains an elevated Cᵢ concentration.

Inside the photobioreactor, the Cᵢ concentration depends on a balance of the rate of CO₂ and HCO₃⁻ utilization by PCC6803 and the Cᵢ supply rate from the membrane carbonation module. Proper control of the Cᵢ delivery rate from the module should enable efficient transfer of CO₂ for growing PCC6803 biomass in the main photobioreactor, controlling the pH, and minimizing CO₂ off-gassing.

To build the membrane carbonation module photobioreactor, a bench-top photobioreactor was integrated with a small module of hollow-fiber membranes, similar to that used for a bench-scale membrane film bioreactor (MBfR).

### Dimensions and Specifications of a Membrane Carbonation Module Photobioreactor

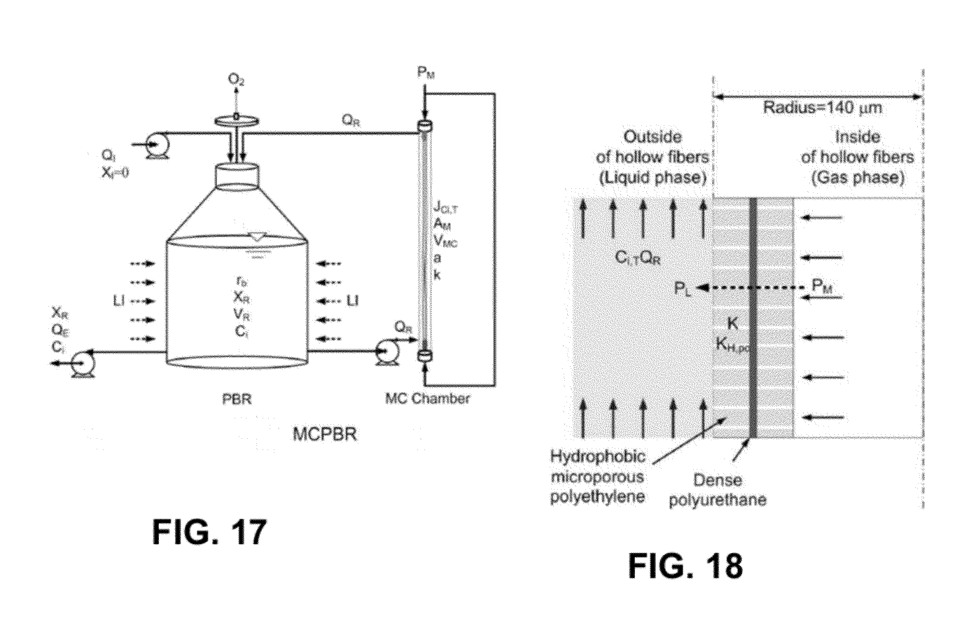

FIG. 17 is a schematic of the membrane carbonation module photobioreactor, and FIG. 18 shows a longitudinal cross-section of a hollow fiber membrane installed in the module of membrane carbonation module chamber. Physical characteristics of the membrane carbonation (MC) module photobioreactor (PBR) used in the proof of concept are shown in Table 1 below.

TABLE 1
Physical characteristics of the membrane carbonation
module photobioreactor used in the proof of concept

| | | |
|---|---|---|
| Item | Unit | Value |

MC

| | | |
|---|---|---|
| Number of hollow fibers | | 25 |
| Membrane surface area, A_{M} | cm² | 44 |
| Specific surface area, a | m⁻¹ | 14,285 |
| Membrane mass transport | Mol/m²/atm/d | 240 |
| Working volume of MC, V_{MC} | L | 0.008 |
| Recirculation flow rate of MC, Q_{R} | L/d | 24, 50 or 73 |

PBR

| | | |
|---|---|---|
| Working volume of PBR, V_{R} | L | 5.5 |
| Influent and effluent flow rate of PBR, | L/d | 1.1 |
| Q_{I} and Q_{E} | | |
| Mixing rate of PBR | rpm | 300 |

The membrane carbonation module photobioreactor consisted of a tubular main reactor made of glass (KIMAX, Germany), a membrane carbonation module chamber, a magnetic stirrer with a spin bar (300 rpm), two peristaltic pumps for influent/effluent and recirculation, two light panels for irradiation, connection tubing including sampling ports, and a gas (O₂) exchange membrane filter to prevent pressure build-up. A laboratory-scale MBfR reactor was modified to be the membrane carbonation module. The glass tube of membrane carbonation module contained a main bundle of composite hollow fiber membranes (model MHF 200TL, Mitsubishi Rayon). The membrane carbonation module fibers were connected to a CO₂ supply tank with Norprene® tubing (Masterflex, USA), plastic barbed fittings, and gastight rubber seals in both ends to guarantee a gastight condition. The CO₂ pressure was constantly controlled by two regulators (3471-A, Matheson Tri-Gas Inc.; Victor HPT100-80-20-BV, Thermadyne Lie).

Two light panels with white-fluorescent lamps (F15T8-RS-CW, General Electric) were placed on both sides of the photobioreactor to supply photosynthetically active radiation (PAR) with a constant illumination level of 44 W/m² each to the exterior photobioreactor surface. A sampling port was installed in the effluent tubing line. The membrane carbonation module was exposed to room light, which had an intensity of approximately 3 W/m² as PAR. The membrane carbonation module photobioreactor had a continuous influent and effluent flow rate that was independent of the internal recirculation rate.

### Inoculum and Culture Media

The membrane carbonation module photobioreactor was inoculated with PCC6803 taken from a mother culture grown in a 10-L glass reservoir bottle (KIMAX, Kimble Chase) aerated with filtered air (2 L_{liquid}/min). The bottle was continuously illuminated using fluorescent lamps (20 W/m² on the exterior) in the photo-incubator chamber (TC30, Conviron Inc.) maintained at 30° C. Non-limiting inorganic nitrogen (Nᵢ, phosphorus (Pᵢ) and other nutrients were supplied using a standard BG-11 with additional Pᵢ using a semi-batch mode of operation (hydraulic retention time≅10 d). To ensure a nutrient-sufficient condition, a modified BG-11 was prepared, containing five times the Pᵢ concentration and no Cᵢ concentration of the original recipe. The medium's total alkalinity was 1.8 meq/L (=90 mg/L as CaCO₃). For all the medium solutions, ultra-pure deionized water (18.2 MΩ-cm) was used, such as that produced by the Purelab Ultra (ELGA LabWater, USA). The mediums were autoclaved before use.

### Start-Up and Operating Procedures of the Membrane Carbonation Module Photobioreactor

The membrane carbonation module photobioreactor was inoculated with 5.5 L of inoculum and then supplied pure CO₂ gas to the membrane carbonation module at a pressure of 15 psi (=103 kPa≅1 atm) and liquid recirculation was started at 24 L/d. The membrane carbonation module photobioreactor was operated with continuous flow and a hydraulic retention time (HRT) of 5 d; the corresponding flow rate of BG-11 medium was 1.1 L/d. At least two volumes of HRT turnover were allowed before the liquid recirculation rate was changed to 50 L/d and then 75 L/d.

### Sampling and Analytical Methods

Operating performance of the membrane carbonation module photobioreactor was monitored by analyzing samples taken from the effluent according to a set sampling plan. One sample per day was taken. All physical, chemical, and biological analyses were determined in duplicate and expressed as average values after appropriate pretreatment and storage at 4° C. To represent global steady state at each flow rate, the last three days of data for all the parameters were averaged.

After filtering samples through a 0.2-μm membrane filter (GD/X, Whatmann, USA), the filtrate was analyzed for anions (NO₃⁻, SO₄²⁻, and PO₄³⁻) and cations (Na⁺, K⁺, Ca²⁺, Mg²⁺, and NH₄⁺) using an ion chromatograph (ICS-3000, Dionex, USA) equipped with IonPac AS18 (Dionex, USA) anion exchange column and CS18 (Dionex, USA) cation exchange column, respectively. Optical density (OD), pH, total Cᵢ, and the concentrations of all carbonate species (i.e., CO_{2(aq)} HCO₃⁻, and CO₃²⁻) were measured, and total alkalinity of modified BG-11 was calculated using the analytical definition of alkalinity, that includes HPO₄²⁻, H⁺, and OH⁻, and converted it to equivalent concentration as CaCO₃.

### Mass Balances for Q and Biomass in Membrane Carbonation Module Photobioreactor

Steady-state mass balances for Cᵢ and biomass in the membrane carbonation module photobioreactor were developed according to Equations 1-4 (below), which are based on the volumes and flows in FIG. 17.

Equation 1 describes the steady-state mass balance for Cᵢ in a membrane carbonation module photobioreactor: Cᵢ supplied from the hollow fiber membranes is balanced by the Cᵢ-uptake reaction for biomass synthesis and Cᵢ loss to the effluent:

J_{CiT}·A_{M}=λ·r_{b}·V+Q_{E}·C_{i,T} (1)

where J_{CiT} is the total Cᵢ flux transferred from the membrane into the liquid (molC/m²/d), A_{M} is the membrane surface area (0.0044 m²=44 cm²), λ is the stoichiometric uptake ratio of Cᵢ to biomass as dry weight (0.51 gC/gDW), r_{b} is the volumetric net biomass production rate as dry weight (g DW/m³/d), V_{R} is the volume of the reactor (0.0055 m³=5.5 L), Q_{E} is the effluent flow rate (=Q₁) (m³/d), and C_{i,T} is the total molar concentration of Cᵢ species (molC/L). An influent mass flow is not included, because the medium contained no inorganic C.

The steady-state mass balance for PCC6803 biomass in a completely stirred tank reactor (CSTR) is described by Equation 2, where X_{R} is the biomass concentration in the membrane carbonation module photobioreactor and its effluent (g DW/m³):

Q_{E}·X_{R}=r_{b}·V (2).

The gradient of CO₂ between inside the membrane and the liquid in the membrane carbonation module promotes diffusion by Fick's law:

J_{CiT}=K·(P_{M}−P_{L}) (3)

where K is the CO₂ mass-transport coefficient for the Mitsubishi hollow fiber membrane 200L membranes (mol/m²/atm/d), P_{M} is the CO_{2(g)} in the hollow fiber membrane module inside (atm), P_{L} is the CO_{2(aq)} in equilibrium with Cᵢ in the liquid (atm), K_{H,cp} is the Henry's Law constant (0.0294 m³·mol/atm), and C_{i,T} is the molar concentration of total Cᵢ (mol/L).

Separate mass balances for steady-state Cᵢ transfer from the membrane to the photobioreactor were developed using two-film theory of gas transfer to liquid. It was assumed that the difference of partial pressure between CO_{2(g)} inside the membrane and CO_{2(aq)} in the liquid drives the mass transport of CO₂ across the membrane wall. The liquid circulating through the membrane carbonation module gains the maximum amount of Cᵢ possible for a given pH, and the transfer rate from the hollow fiber membrane is not limiting. Since recirculation transfers newly supplied CO_{2(aq)} to the main photobioreactor, the transfer rate is the same as the rate at which CO_{2(aq)} (≅H₂CO₃*) diffuses through membrane, as shown in Equation 4:

ΔC_{i,T}·Q_{R}=K·A_{M}·(P_{M}−P_{L})=K·A_{M}·(P_{M}−α₀·C_{i,T}K_{H,pc}) (4)

where Q_{R} is the recirculation flow rate passing through membrane carbonation module (m³/d), ΔC_{i,T} is the difference between influent and effluent C_{i,T} of membrane carbonation module, and α₀ is the fractional ionization constant for CO_{2(aq)} depending on liquid pH.

Equation 1 equals Equation 4 at global steady-state, because CO_{2(g)} that diffuses through the membrane is balanced by Cᵢ invested for biomass synthesis and lost in the effluent, keeping the C_{i,T} concentration stable. The uptake of CO_{2(aq)} and HCO₃⁻ for synthesis is related to the rate of biomass synthesis by stoichiometry (i.e., in Equation 1).

### Results and Discussion

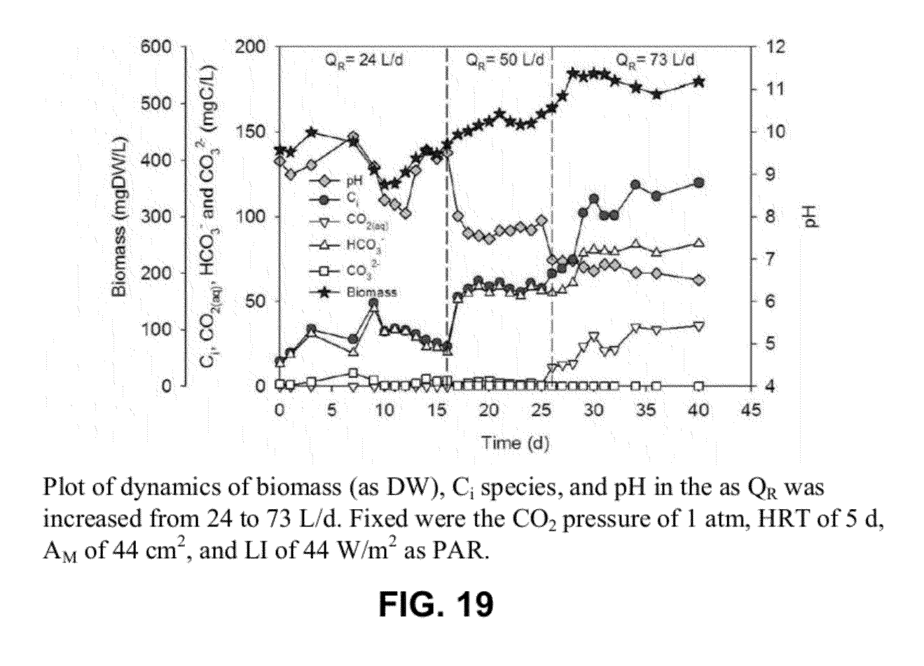

The membrane carbonation module photobioreactor was operated in a continuous mode for harvesting biomass and recharging with fresh medium. Based on daily samples, FIG. 19 shows the concentrations of biomass and soluble components for the three recirculation flow rates: Q_{R}=24, 50, and 73 L/d. Increasing Q_{R} from 24 to 73 L/d, led to higher biomass concentrations as DW: from 420 mgDW/L to 528 mgDW/L. Each Q_{R} achieved a steady biomass concentration within about 10 days, and this gave a specific growth rate (μ=Q/V) of 0.2 d⁻¹, assuming that all the biomass was suspended. Thus, increasing Q_{R} provided a higher Cᵢ-delivery rate that allowed more biomass accumulation.

FIG. 19 also presents the Cᵢ, CO_{2(aq)}, HCO₃⁻, CO₃²⁻, and pH values. For days 0 to 16, when Q_{R} was 24 L/d, total Cᵢ averaged 25 mgC/L, except for a transient increase to 50 mgC/L on day 9. The average pH of 9.5 made HCO₃⁻ the dominant Cᵢ species at 22 mgC/L; CO₃²⁻ was only 3 mgC/L, and CO_{2(aq)} was negligible. From day 16, Q_{R} (to 50 L/d) was approximately doubled, which led to an increase of average Cᵢ to ˜62 mgC/L. Since the average pH declined to 7.6, HCO₃⁻ became ˜95% of Cᵢ, with CO_{2(aq)} approximately 5% and CO₃²⁻ negligible. When the flow rate Q_{R} was further increased to 73 L/d, the steady-state Cᵢ reached as high as 117 mgC/L, and the average Cᵢ was 98 mgC/L, which is 1.7-fold higher than for 50 L/d. With the pH declining to 6.7, the molar ratio between CO_{2(aq)} and HCO₃⁻ increased to 3:7. The K_{S} for Cᵢ, including CO_{2(aq)} and HCO₃⁻ (2% and 98% for those conditions), is about 0.5 mgC/L for PCC6803 at pH 8; therefore, PCC6803 in the membrane carbonation module photobioreactor should have experienced no Cᵢ limitation. In addition, none of NO₃⁻, SO₄²⁻, and PO₄³⁻ was present at a rate-limiting concentration.

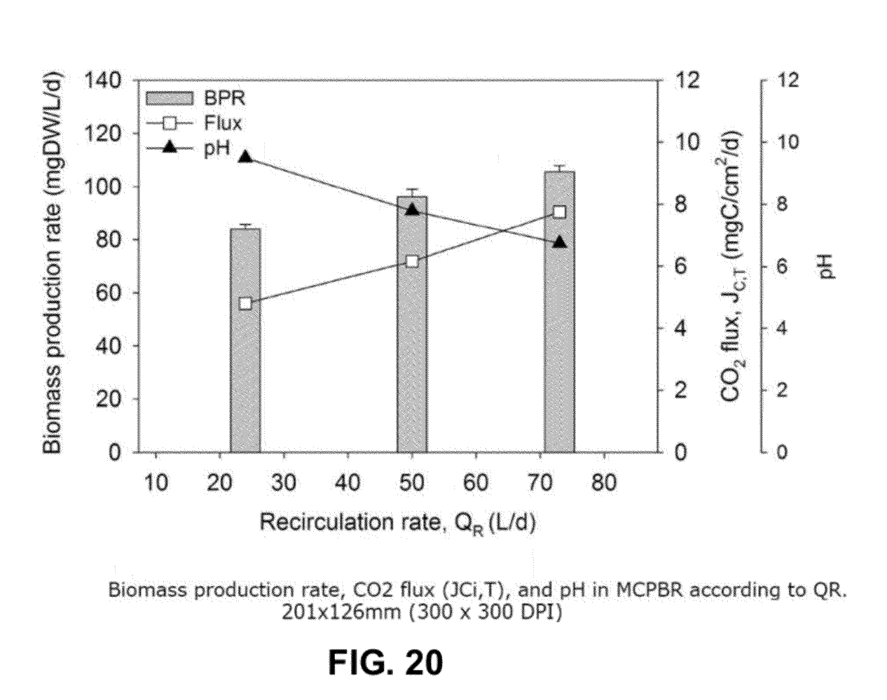

The clear correlation of Cᵢ and pH to Q_{R} as shown in FIG. 20 demonstrates that the membrane carbonation module photobioreactor achieved the goal of managing the rate of photosynthesis by controlling the Cᵢ-delivery rate. The relationship was not linear, as Cᵢ increased proportionally more than did Q_{R}: for the Q_{R} ratios of 1.0:2.1:3.0, the Cᵢ ratios were 1.0:2.5:4.6.

FIG. 20 compares the CO₂ flux, the biomass production rate, and the pH at each operating condition. The flux was computed using the effluent flow rate, the measured Cᵢ concentration, and the stoichiometric utilization of Cᵢ according to Equations 1 and 2. The biomass production rate was computed from Equation 2. Increasing Q_{R} from 24 to 73 L/d improved the total delivery of CO_{2(g)} to the liquid from 5 to 8 mgC/cm²/d, and that resulted in increases of the biomass production rate: from 84 mg DW/Ld with Q_{R}=24 L/d to 106 mg DW/L/d for Q_{R}=74 L/d. This demonstrates that the biomass production rate can be managed by adjusting Q_{R} when other conditions are held constant (i.e., CO₂ pressure=1 atm, A_{M}=44.0 cm², μ=0.2 d⁻¹, and light irradiance=44 W/m² as PAR).

In the membrane carbonation module photobioreactor, the high pH at Q_{R}=24 L/d resulted from relatively insufficient CO₂ delivery from the membrane carbonation module due to photoautotrophic consumption of HCO₃⁻ and CO_{2(aq)}. As Q_{R} increased to Q_{R}=50 L/d and 73 L/d, however, total Cᵢ delivery improved from 6.1 mgC/cm²/d (Q_{R}=24 L/d) to 7.6 and 9.2 mgC/cm²/d, respectively, resulting in more abundant steady-state Cᵢ and a pH decrease inside membrane carbonation module photobioreactor. Thus, proper adjustment of Q_{R} (from 24 to 50 L/d) provided a superior pH, since PCC6803 prefers slightly alkaline pH.

Table 2 shows membrane mass transport and CO₂ transfer efficiency based on mass balance during continuous operation of membrane carbonation module photobioreactor.

TABLE 2
Membrane mass transport and CO₂ transfer efficiency

| | | |
|---|---|---|
| Item | Unit | Values |

Independent variable

| | | | | |
|---|---|---|---|---|
| Q_{R} | L/d | 24 | 50 | 73 |

Fixed operating conditions

| | | | | |
|---|---|---|---|---|
| Q_{B} | L/d | 1.1 | 1.1 | 1.1 |
| V | L | 5.5 | 5.5 | 5.5 |
| A_{M} | cm² | 44 | 44 | 44 |
| p_{M} | atm | 1.02 | 1.02 | 1.02 |
| K_{μpc} | m³ · atm/mol | 0.0294 | 0.0294 | 0.0294 |
| Re^{a} | | 60 | 124 | 180 |

Stoichiometry

| | | | | |
|---|---|---|---|---|
| λ | mg C/mg DW | 0.51 | 0.51 | 0.51 |

Experimental measures

| | | | | |
|---|---|---|---|---|
| Cᵢ | g C/m³ | 29.3 | 59.0 | 97.6 |
| C_{i, T} | mol C/m³ | 2.4 | 4.9 | 8.1 |
| X_{E} | mg DW/L | 420 | 480 | 528 |
| λX_{E} | g C/m³ | 215 | 246 | 270 |
| | mol C/m³ | 18 | 20 | 23 |
| pH | | 9.1 | 7.6 | 6.8 |

Estimated variables

| | | | | |
|---|---|---|---|---|
| K | mol/m²/atm/d | 5.0 | 6.3 | 7.9 |
| J_{Ci, T} | mg C/cm²/d | 6.1 | 7.6 | 9.2 |
| J_{Ci, T} A_{M}/V | g C/m³/d | 49 | 61 | 73 |
| α₀ | | 0.0016 | 0.0436 | 0.2315 |

^{a}Reynolds number (Re) for the flow in the MC = Q_{R}D_{H}/νA. We assumed that the kinematic viscosity (ν) is the same as for water (0.8 × 10⁻⁶ m²/s) at 30° C., and the hydraulic diameter (D_{H}) and cross-sectional area of the MC were 0.006 m and 2.8 × 10⁻⁵ m², respectively.

Table 2 shows estimated values for the CO_{2(aq)} fractional ionization constant (α₀), mass-transfer flux, volumetric mass-transfer rate, and K-estimates for each condition; these values were computed using mass balance equations 1-4 and the operating and measured values given in the upper parts of the table.

Three significant and related trends are revealed. First, the three measures of mass transport via the hollow fiber membrane surface increased from 5.0 to 7.9 mol/m²/atm/d as Q_{R} increased, but not in linear proportion to Q_{R}. This trend corresponds to literature indicating that increased water velocity past the fibers promotes CO₂ mass transport in hollow fiber membranes due to improved liquid-side mass-transport. The increase in liquid-side transport is proportional to Reynolds number (Re). The Re in membrane carbonation module chamber increased from 60 to 180. This increase in mass-transport kinetics in the membrane carbonation module is an extra benefit from increasing Q_{R}. Due to the increased advection of Cᵢ and faster mass transport, higher Re (and Q_{R}) improved the volumetric Cᵢ delivery rate to the photobioreactor (up to ˜73 gC/m³/d).

Second, the higher Cᵢ delivery rate allowed the Cᵢ concentration in the photobioreactor to increase, lowered the pH, and caused α₀ to become larger. This underscores that adjusting Q_{R} is a means to maintain adequate Cᵢ and pH. The relationship between Cᵢ and pH depends on the alkalinity in the medium, 90 mg/L as CaCO₃. With alkalinity fixed in the influent, the membrane carbonation module allowed us control over pH by delivering different amounts of Cᵢ. A different total alkalinity would change the relationship among J_{Ci,T}, Cᵢ, and pH.

Last, the availability of light irradiance, the sole energy source for photosynthetic activity, eventually controlled the degree to which the biomass concentration could be increased by increasing Q_{R}. For example, increasing Q_{R} 1.5-fold (50 to 73 L/d) gave an increase in the biomass concentration of only 1.1-fold, while C_{i,T} increased 1.7-fold. Nutrient limitation was not a factor, so light limitation affected the growth kinetics. With the biomass synthesis rate increasing proportionally less than the increase in Cᵢ delivery rate, Cᵢ increased (from ˜29 to ˜98 gC/m³), pH decreased (from ˜9.1 to ˜6.7), and α₀ increased (from ˜0.002 to ˜0.23).

In summary, the membrane carbonation module photobioreactor can manage the biomass-production rate by controlling the CO₂ transfer rate. The CO₂ transfer rate was controlled by the recirculation flow rate Q_{R}. The membrane carbonation module photobioreactor approach offers the advantage of independent control over photoautotrophic growth kinetics, Cᵢ, and pH, while minimizing off-gas CO₂ by avoiding aeration. Possible additional components and practices could include an increase of A_{M} to improve mass transport of CO₂, which results in increase of total Cᵢ-supply to photobioreactor. Also, there can be the application of higher light irradiance to overcome the declining benefits of a high Q_{R} control. Alkalinity concentration can be controlled to optimize the relationship between the Cᵢ delivery rate and pH. The ratio of V_{R} to V_{MC} can be adjusted to optimize the overall volumetric productivity of biomass.

What is claimed is:

1. A system comprising:
a photobioreactor comprising a vessel comprising a center and light-permitting wall; and
a membrane carbonation module within the photobioreactor comprising a plurality of hollow fiber membranes, each hollow fiber membrane comprising a membrane wall forming an inner lumen.

2. The system of claim 1, wherein during use, the photobioreactor comprises a liquid and photoautotrophic microorganisms suspended in the liquid and the membrane carbonation module is in operable contact with the liquid.

3. The system of claim 1, wherein the photoautotrophic microorganisms are cyanobacteria.

4. The system of claim 1, further comprising a pressure modulator coupled to the membrane carbonation module.

5. The system of claim 4, where each hollow fiber membrane is sealed at one end and the pressure modulator is configured to supply CO₂ to the inner lumens of the hollow fiber membranes during use.

6. The system of claim 4, where the pressure modulator is configured to apply negative pressure to the inner lumen of each hollow fiber membrane during use.

7. The system of claim 1, where the membrane carbonation module is positioned within the vessel, and the photobioreactor comprises a light region near the light-permitting wall and a dark region near the center.

8. The system of claim 7, where the membrane carbonation module is positioned within the light region.

9. The system of claim 7, where the membrane carbonation module is positioned within the dark region.

10. The system of claim 1, further comprising a recirculation chamber coupled to the photobioreactor and a pump coupled to the photobioreactor and the recirculation chamber, where the pump is configured to circulate a volume of liquid from the vessel, to the recirculation chamber, and back to the vessel during use.

11. The system of claim 10, where the membrane carbonation module is positioned within the recirculation chamber.

12. The system of claim 1, further comprising a plurality of membrane carbonation modules.

13. The system of claim 12, where at least one membrane carbonation module is positioned within the vessel, and the photobioreactor comprises a light region near the light-permitting wall and a dark region near the center.

14. The system of claim 13, where at least one membrane carbonation module is positioned within the light region.

15. The system of claim 13, where at least one membrane carbonation module is positioned within the dark region.

16. The system of claim 1, further comprising:
at least one recirculation chamber coupled to the photobioreactor; and
a pump coupled to the photobioreactor, where the pump is configured to circulate a volume of liquid from the vessel, to the recirculation chamber, and back to the vessel during use.

17. The system of claim 16, where at least one membrane carbonation module is positioned within the at least one recirculation chamber.

18. The system of claim 1, further comprising a plurality of pressure modulators, where each pressure modulator is coupled to a corresponding membrane carbonation module.

19. The system of claim 18, where at least one pressure modulator is configured to supply CO₂. to the inner lumens of the hollow fiber membranes.

20. The system of claim 18, where at least one pressure modulator is configured to apply negative pressure to the inner lumens of the hollow fiber membranes of one membrane carbonation module.

21. A method comprising:
placing a liquid and photoautotrophic microorganisms in the photobioreactor of a system of claim 1; and
diffusing gas molecules across the membrane walls of the plurality of hollow fiber membranes.

22. The method of claim 21, where the system further comprises a pressure modulator coupled to the membrane carbonation module.

23. The method of claim 22, further comprising supplying CO₂ gas to the plurality of hollow fiber membranes using the pressure modulator and diffusing CO₂ molecules across each membrane wall from the inner lumen to the liquid.

24. The method of claim 23, further comprising adjusting the rate at which CO₂ gas is supplied to the plurality of hollow fiber membranes until a desired pH level is reached.

25. The method of claim 21, where the membrane carbonation module is positioned within the vessel, and the photobioreactor comprises a light region near the light-permitting wall and a dark region near the center.

26. The method of claim 25, where the membrane carbonation module is positioned in the light region.

27. The method of claim 25, where the membrane carbonation module is positioned in the dark region.

28. The method of claim 21, further comprising diffusing a gaseous product through each membrane wall from the liquid to the inner lumens of the hollow fiber membranes.

29. The method of claim 28, further comprising applying a negative pressure to the plurality of hollow fiber membranes with the pressure modulator and collecting the gaseous product.

30. The method of claim 29, where the gaseous product is H₂, O₂, or N₂.
